(19) United States
(12) Patent Application Publication
(10) Pub. No.: US 2026/0356990 A1
(43) Pub. Date: Nov. 12, 2026

(54) HEPARIN DODECASACCHARIDES CONTAINING TWO ANTITHROMBIN-BINDING SEQUENCES AS WELL AS PREPARATION METHODS AND APPLICATIONS THEREOF

(71) Applicant: SHANDONG UNIVERSITY, Jinan (CN)

(72) Inventors: Chunhui LIU, Jinan (CN); Guijiao ZHANG, Jinan (CN); Yaqi QIU, Jinan (CN); Lin WANG, Jinan (CN); Jingru LI, Jinan (CN)

(73) Assignee: SHANDONG UNIVERSITY, Jinan (CN)

(21) Appl. No.: 19/102,425

(22) PCT Filed: Aug. 8, 2023
(86) PCT No.: PCT/CN2023/111623

§ 371 Date: Feb. 10, 2025

(30) Foreign Application Priority Data
Aug. 12, 2022 (CN) 202210971148.4

(51) Int. Cl. C08B 37/00; A61K 31/727; A61P 7/02; C12N 1/20; C12N 9/10; C12N 9/90; C12P 19/18; C12P 19/24; C12P 19/26; C12R 1/19

(52) U.S. Cl. CPC C08B 37/0075; A61K 31/727; A61P 7/02; C12N 1/20; C12N 9/1051; C12N 9/13; C12N 9/90; C12P 19/18; C12P 19/24; C12P 19/26; C12Y 208/02008; C12Y 501/03017; C12R 2001/19

(57) ABSTRACT

The present disclosure relates to heparin dodecasaccharides containing two antithrombin-binding sequences as well as preparation methods and applications thereof, having the structure represented by formula I. The disclosure also provides chemoenzymatic preparation methods and applications for compounds containing the structure in formula I. The heparin dodecasaccharides of the disclosure are synthesized with significantly fewer steps and a notably higher overall yield. They exhibit potent and specific anti-Factor Xa activity, which can be effectively neutralized by protamine with a neutralization rate of >80%. The absence of consecutive multiple trisulfated disaccharide units (IdoA2S-GlcNS6S) avoids pharmacokinetic deficiencies (e.g., short half-life) and adverse reactions (e.g., heparin-induced thrombocytopenia, HIT) associated with such sequences. The heparin dodecasaccharides are suitable for the preparation of safer and more cost-effective novel anticoagulant and antithrombotic drugs.

### CROSS-REFERENCE TO RELATED APPLICATIONS

This application is the U.S. national phase of International Application No. PCT/CN2023/111623 filed on 8 Aug. 2023 which designated the U.S. and claims priority to Chinese Application No. CN 202210971148.4 filed on 12 Aug. 2022, the entire contents of each of which are hereby incorporated by reference.

### TECHNICAL FIELD

The present disclosure relates to heparin dodecasaccharides containing two antithrombin-binding sequences as well as a preparation methods and applications thereof, belonging to the technical field of biomedicine.

### BACKGROUND

Heparin-based drugs have been used as clinical anticoagulants for over 90 years and remain widely utilized in the treatment of thromboembolic diseases, surgery, hemodialysis, with a global market size of exceeding US$8 billion. Currently marketed unfractionated heparin (UFH), primarily extracted from porcine intestinal mucosa, is a polydisperse mixture of polysaccharide with an average molecular weight of ~14,000 Da. The low-molecular-weight heparins (LMWHs), such as enoxaparin, dalteparin, nadroparin, and tinzaparin, are prepared through controlled chemical or enzymatic depolymerization of UFH and represent complex low-molecular-weight mixtures with average molecular weights typically ranging from 3,500 to 6,000 Da. LMWHs now have become the preferred clinical anticoagulants. While animal-derived UFH and LMWH offer advantages such as relatively low cost and robust production processes, however, their structural heterogeneity results in inherent clinical limitations and issues of contamination and fragile supply chains. Research has demonstrated that the anticoagulant activity of animal-derived heparin highly depends on a unique pentasaccharide sequence (abbreviated as: GlcNS/Ac6S-GlcA-GlcNS6S3S-IdoA2S-GlcNS6S) that specifically binds to antithrombin (AT). The sequence is randomly distributed along the polysaccharide chains and occurs in approximately one-third of heparin molecule. Fondaparinux sodium (trade name Arixtra), a methyl glycoside derivative of this pentasaccharide sequence, was approved for marketing in 2001 and became the first fully synthetic, structurally defined heparin-based single compound, paving the way for the development of next-generation anticoagulant heparin drugs.

Protamine is the first FDA-approved antidote for animal-derived heparin, which can eliminate the anticoagulant effects of heparin to restore normal coagulation in the body. Protamine can completely neutralizes the anticoagulant effect of UFH, but only partially neutralizes the effect of LMWHs. This “neutralizable” characteristic enables the timely termination of anticoagulant therapy according to clinical requirements, effectively reducing the risk of bleeding and other adverse effects. Unfortunately, the synthetic fondaparinux is not neutralized by protamine, significantly limiting its clinical application. Therefore, it is highly desirable to discover new anticoagulant heparin molecules that can be neutralized by protamine.

Professor Jian Liu of the University of North Carolina at Chapel Hill reported a synthetic heparin dodecasaccharide containing a single AT-binding pentasaccharide and four consecutive trisulfated disaccharide (IdoA2S-GlcNS6S) units using a chemoenzymatic approach. Its anticoagulant activity was effectively neutralized by protamine. However, the synthesis involved 22 to 23 tedious steps, resulting in a relatively low overall yield. Moreover, the presence of four consecutive trisulfated disaccharide units may lead to undesirable pharmacokinetic profiles such as a short half-life in vivo and potential adverse effects like heparin-induced thrombocytopenia (HIT). Therefore, there is an urgent need to develop new “neutralizable” anticoagulant heparin-based molecules with higher anticoagulant efficiency, improved pharmacokinetics, and fewer complications.

### SUMMARY OF THE INVENTION

In view of the limitations of the prior art, the present disclosure provides heparin dodecasaccharides containing two antithrombin-binding sequences as well as preparation methods and applications thereof.

### DESCRIPTION OF TERMS

AT: antithrombin
IdoA: iduronic acid
GlcA: glucuronic acid
UDP-GlcNTFA: uridine diphosphate-N-trifluoroacetylglucosamine
UDP-GlcNAc: uridine diphosphate-N-acetylglucosamine
UDP-GlcA: uridine diphosphate-glucuronic acid
PAPS: 3′-phosphoadenosine-5′-phosphosulfate
KfiA: Escherichia coli K5 N-acetylglucosaminyltransferase
PmHS2: Pasteurella multocida Heparosan synthase 2
NST: N-sulfotransferase
C₅-epi: C₅-isomerase
2OST: 2-O-sulfotransferase
6OST: 6-O-sulfotransferase
3OST: 3-O-sulfotransferase

The present disclosure is implemented by the following technical solutions.

The first object of the present disclosure is to provide heparin dodecasaccharide molecules containing two AT-binding sequences and devoid of multiple consecutive trisulfated disaccharide (IdoA2S-GlcNS6S) units or a pharmaceutically acceptable salt thereof, having the structure shown in formula I as follows:

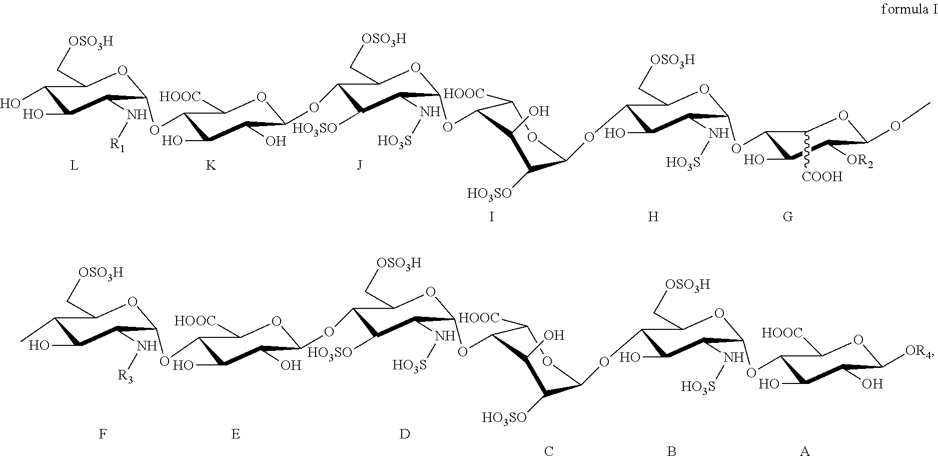

Where R₁ and R₃ are sulfo groups (—SO₃H) or acetyl groups (—COCH₃); R₂ is sulfo groups (—SO₃H) or hydrogen (—H);
R₄ is selected from phenyl or substituted phenyl group with characteristic UV absorption, or aromatic heterocycle or substituted aromatic heterocycle;
Sugar residue G is glucuronic acid (GlcA) or iduronic acid (IdoA) residue.

According to a preferred embodiment of the present disclosure, the substituents on the substituted phenyl or substituted aromatic heterocycle are nitro, halogen, hydroxyl, or trifluoromethyl groups.

According to a preferred embodiment of the present disclosure, a novel heparin dodecasaccharide containing two AT-binding sequences and devoid of multiple consecutive trisulfated disaccharide units is selected from any one of the following compounds or pharmaceutically acceptable salts thereof:

| No. | Residue G | R₁~R₄ |
|---|---|---|
| I-1 | GlcA | R₁═R₃═—SO₃H, R₂═H, R₄═—PNP |
| I-2 | GlcA | R₁═—SO₃H, R₃═—Ac, R₂═H, R₄═—PNP |
| I-3 | GlcA | R₁═—Ac, R₃═—SO₃H, R₂═H, R₄═—PNP |
| I-4 | GlcA | R₁═R₃═—Ac, R₂═—H, R₄═—PNP |
| I-5 | GlcA | R₁═R₂═R₃═—SO₃H, R₄═—PNP |
| I-6 | GlcA | R₁═R₂═—SO₃H, R₃—Ac, R₄═—PNP |
| I-7 | GlcA | R₁═—Ac, R₂=R₃═—SO₃H, R₄═—PNP |
| I-8 | GlcA | R₁═R₃═—Ac, R₂═—SO₃H, R₄═—PNP |
| I-9 | IdoA | R₁═R₂═R₃═—SO₃H, R₄═—PNP |
| I-10 | IdoA | R₁═R₂═SO₃H, R₃═—Ac, R₄═—PNP |
| I-11 | IdoA | R₁═—Ac, R₂=R₃═—SO₃H, R₄═—PNP |
| I-12 | IdoA | R₁═R₃═—Ac, R₂═—SO₃H, R₄═—PNP |

According to a preferred embodiment of the present disclosure, a heparin dodecasaccharide containing two AT-binding sequences and devoid of multiple consecutive trisulfated disaccharide (IdoA2S-GlcNS6S) units exhibits significant anti-factor Xa activity, with no significant anti-factor IIa activity. The anti-factor Xa activity can be effectively neutralized by protamine, with a neutralization rate of >80%.

The second objective of the present disclosure is to provide a preparation method of the heparin dodecasaccharide containing two AT-binding sequences and devoid of multiple consecutive trisulfated disaccharide (IdoA2S-GlcNS6S) units, using a chemoenzymatic synthesis strategy.

The preparation method of the heparin dodecasaccharide containing two AT-binding sequences and devoid of multiple consecutive trisulfated disaccharide units uses a glucuronic acid (GlcA) derivative with a reducing end covalently linked to an R₄ group as the starting substrate and comprises the following steps: the glycosyltransferase-catalyzed reactions (steps a and b) repeated at least one once, and a combination with four or five steps selected from chemoenzymatic modification reactions (steps c, d, e, f, and g);
at step a: under the catalysis of N-acetylglucosaminyltransferase (KfiA) or Heparosan synthase 2 (PmHS2), using UDP-GlcNTFA or UDP-GlcNAc as the glycosyl donor, the GlcNTFA or GlcNAc residue of the glycosyl donor is transferred to GlcA at the non-reducing end of the substrate via an α-1,4-glycosidic bond, yielding an intermediate compound;
at step b: under the catalysis of PmHS2 enzyme, using UDP-GlcA as the glycosyl donor, the GlcA residue of the glycosyl donor is linked to glucosamine (GlcNTFA or GlcNAc) at the non-reducing end of the substrate via a β-1,4-glycosidic bond, yielding an intermediate compound;
at step c: the heparin intermediate is incubated in a mild alkaline aqueous solution on ice, where all the GlcNTFA residues of the sugar chain are completely N-detrifluoroacetylated to form GlcNH₂, subsequently these GlcNH₂ residues are converted to GlcNS under the catalysis of N-sulfotransferase (NST), yielding an N-sulfated intermediate;
at step d: under the simultaneous catalysis of C₅-epimerase (C₅-epi) and 2-O-sulfotransferase (2OST), a specific GlcA residue flanked between two GlcNS residues or between GlcNS (at the non-reducing end) and GlcNAc in the N-sulfated product is converted to 2-O-sulfated iduronic acid (IdoA2S), yielding an intermediate containing an IdoA2S residue;
at step e: under the catalysis of 2OST alone, a specific GlcA residue flanked between two GlcNS residues or between GlcNS (at the non-reducing end) and GlcNAc in the substrate is converted to 2-O-sulfated glucuronic acid (GlcA2S), yielding an intermediate containing GlcA2S residues;
at step f: under the co-catalysis of 6-O-sulfotransferase 1 and 3 (6OST1, 6OST3), all GlcNS or GlcNAc residues in the sugar chain of the substrate are sulfated at the 6-OH position to form GlcNS6S or GlcNAc6S residues, resulting in a 6-O-sulfated intermediate; and
at step g: under the catalysis of 3-O-sulfotransferase 1 (3OST1), the 3-OH group of a GlcNS6S between GlcA and IdoA2S in the sugar chain of the substrate is 3-O-sulfated (GlcNS6S3S), yielding the final target compound.

According to a preferred embodiment of the present disclosure, the starting substrate is p-nitrophenyl-β-D-glucuronide (GlcA-PNP).

According to a preferred embodiment of the present disclosure, at step a, the recombinant N-acetylglucosaminyltransferase (KfiA) and Heparosan synthase 2 (PmHS2) are expressed in Escherichia coli; KfiA is derived from Escherichia coli K5, and PmHS2 is derived from Pasteurella multocida.

According to a preferred embodiment of the present disclosure, at steps a and b, the enzymatic reactions are carried out at 20 °C to 37 °C in 50 mmol/L Tris-HCl buffer solution containing 6 mmol/L MnCl₂, pH 7.0-7.5. The amounts of enzyme and substrate added, as well as the reaction time, are not limited. The resulting enzymatic reaction mixture is purified by reverse-phase C₁₈ or anion-exchange column chromatography to obtain the intermediate compounds.

According to a preferred embodiment of the present disclosure, at steps a and b, the glycosyl donor is added in at least 1.2 equivalents relative to the substrate.

According to a preferred embodiment of the present disclosure, at steps c, d, e, f, and g, the recombinant heparin-modifying enzymes NST, C₅-epi, 2OST, 6OST1, 6-OST3, and 3OST1 are expressed using Escherichia coli, yeast, or insect cells, using 3′-phosphoadenosine-5′-phosphosulfate (PAPS) as the sulfate donor. The enzymatic reactions are carried out at 20 °C to 37 °C in 50 mmol/L 2-(N-morpholino) ethanesulfonic acid (MES) buffer, pH 7.0-7.5. The amounts of enzyme and heparin intermediate substrate added, as well as the reaction time, are not limited. The resulting reaction mixture is purified by anion-exchange column chromatography to obtain the product.

According to a preferred embodiment of the present disclosure, at steps c, d, e, f, and g, the sulfate donor is added in 1.5-10 equivalents of the substrate.

The preparation method of the heparin dodecasaccharide containing two AT-binding sequences according to the present disclosure is established based on extensive experimental research on the catalytic activity and substrate specificity of various glycosyltransferases and heparin-modifying enzymes toward different heparin intermediate molecules.

According to a preferred embodiment of the present disclosure, the preparation method is selected from one of the following synthetic routes:

| No. | synthetic routes |
|---|---|
| I-1 | a→b→a→b→c→d→a→b→c→f→a→b→a→b→c→d→a→c→f→g |
| I-2 | a→b→a→b→c→d→a→b→f→a→b→a→b→c→d→a→c→f→g, or |
| | a→b→a→b→c→d→a→b→a→b→a→b→c→d→a→c→f→g |
| I-3 | a→b→a→b→c→d→a→b→c→f→a→b→a→b→c→d→a→f→g |
| I-4 | a→b→a→b→c→d→a→b→f→a→b→a→b→c→d→a→f→g |
| I-5 | a→b→a→b→c→d→f→a→b→a→b→c→e→a→b→c→d→a→c→f→g |
| I-6 | a→b→a→b→c→d→f→a→b→a→b→c→e→a→b→c→d→a→c→f→g |
| I-7 | a→b→a→b→c→d→f→a→b→a→b→c→e→a→b→c→d→a→f→g |
| I-8 | a→b→a→b→c→d→f→a→b→a→b→c→e→a→b→c→d→a→f→g |
| I-9 | a→b→a→b→c→d→f→a→b→a→b→a→b→c→d→a→c→f→g |
| I-10 | a→b→a→b→c→d→f→a→b→a→b→a→b→c→d→a→c→f→g, OR |
| | a→b→a→b→c→d→a→b→a→b→a→b→c→d→a→c→f→g |
| I-11 | a→b→a→b→c→d→f→a→b→a→b→a→b→c→d→a→f→g |
| I-12 | a→b→a→b→c→d→f→a→b→a→b→a→b→c→d→a→f→g |

Most preferably, the preparation method for the heparin dodecasaccharide containing two AT-binding sequences and devoid of multiple consecutive trisulfated disaccharide units follows the synthetic route below:
a→b→a→b→c→d→a→b→a→b→a→b→c→d→a→c→f→g_{∘}

Specifically, the preparation method for the heparin dodecasaccharide containing two AT-binding sequences and devoid of multiple consecutive trisulfated disaccharides comprises the following steps:
under the catalysis of KfiA or PmHS2, using UDP-GlcNTFA as the glycosyl donor, the GlcNTFA residue of the glycosyl donor is transferred to GlcA at the non-reducing end of the substratevia an α-1,4-glycosidic bond, yielding a disaccharide backbone intermediate;
under the catalysis of PmHS2 enzyme, using UDP-GlcA as the glycosyl donor, the GlcA residue of the glycosyl donor is linked to GlcNTFA at the non-reducing end of the disaccharide backbonevia a β-1,4-glycosidic bond, yielding a trisaccharide backbone intermediate;
repeating steps 1) and 2) to extend the sugar chain, resulting in a pentasaccharide backbone intermediate;
4) The pentasaccharide backbone intermediate is incubated in a mild alkaline aqueous solution on ice, where all the GlcNTFA residues of the sugar chain undergo complete N-detrifluoroacetylation to form GlcNH₂, subsequently, these GlcNH₂ residues are converted to GlcNS under the catalysis of NST, yielding an N-sulfated pentasaccharide intermediate;
5) under the co-catalysis of C5-epi and 2OST, a specific GlcA residue between two GlcNS in the N-sulfated pentasaccharide intermediate is converted to 2-O-sulfated iduronic acid (IdoA2S), yielding a heparin pentasaccharide containing one IdoA2S residue;
6) referring to step 1), the glycosyl donor is replaced with UDP-GlcNAc, and the sugar chain is extended under the catalysis of KfiA or PmHS2 to obtain a hexasaccharide intermediate; using the hexasaccharide intermediate as the substrate, the sugar chain is further extended to a heptasaccharide by referring to step 2), yielding a heptasaccharide intermediate; by alternately repeating steps 1) and 2), the heptasaccharide intermediate substrate is further extended to an undecasaccharide intermediate;
7) the undecasaccharide intermediate is incubated in a mild alkaline aqueous solution on ice, where the GlcNTFA residues of the sugar chain undergo complete N-detrifluoroacetylation to form GlcNH₂; subsequently, these GlcNH₂ residues are converted to GlcNS under the catalysis of NST, yielding an N-sulfated undecasaccharide intermediate;
8) under the co-catalysis of C₅-epi and 2OST, a specific GlcA between two GlcNS in the N-sulfated undecasaccharide is converted to 2-O-sulfated iduronic acid (IdoA2S), resulting in a heparin undecasaccharide containing two IdoA2S residues; by adding an appropriate amount of enzyme and substrate and extending the reaction time, a specific GlcA residue between GlcNS (non-reducing end) and GlcNAc in the undecasaccharide is further converted to IdoA2S, yielding a heparin undecasaccharide containing three IdoA2S residues;
9) in step 7), the heparin undecasaccharides containing two or three IdoA2S residues are subjected to additional step 1) to extend the sugar chain, resulting in heparin dodecasaccharides containing two or three IdoA2S residues;
10) the heparin dodecasaccharides containing two or three IdoA2S residues are incubated in a mild alkaline aqueous solution on ice, where the newly introduced GlcNTFA residues of the sugar chain undergo complete N-detrifluoroacetylation to form GlcNH₂; subsequently, these GlcNH2 residues are converted to GlcNS under the catalysis of NST, yielding N-sulfated heparin dodecasaccharides containing two or three IdoA2S residues;
11) under the co-catalysis of 6-O-sulfotransferase 1 and 3 (6OST1, 6OST3), all GlcNS or GlcNAc residues in the sugar chain of the N-sulfated heparin dodecasaccharides containing two or three IdoA2S residues are sulfated at the 6-OH position to form GlcNS6S or GlcNAc6S, resulting in two 6-O-sulfated heparin dodecasaccharides;
12) under the catalysis of 3-O-sulfotransferase 1 (3OST1), the 3-OH group of GlcNS6S between GlcA and IdoA2S in the sugar chain of the two 6-O-sulfated heparin dodecasaccharides is sulfated (GlcNS6S3S), yielding the target compound I-2 containing two IdoA2S residues or the target compound I-10 containing three IdoA2S residues, respectively.

The third objective of the present disclosure is to provide the application of the heparin dodecasaccharides containing two AT-binding sequences and devoid of multiple consecutive trisulfated disaccharide units for the preparation of anticoagulant and antithrombotic drugs.

An anticoagulant and antithrombotic drug containing the aforementioned heparin dodecasaccharide containing two AT-binding sequences and devoid of multiple consecutive trisulfated disaccharides, along with one or more pharmaceutically acceptable carriers or excipients, wherein the ratio of the heparin dodecasaccharide to the carrier or excipient is not limited.

The present disclosure has the following technical features and advantages:
1. Based on the chromogenic substrate assay, the novel heparin dodecasaccharide of the present disclosure, containing two AT-binding sequences and devoid of multiple consecutive trisulfated disaccharide units, exhibits significant anti-factor Xa activity without notable anti-factor IIa activity. Furthermore, this inventiondemonstrate for the first time that the two AT-binding pentasaccharide sequences linked by GlcA/IdoA can independently activate AT, resulting in potent inactivation effects on factor Xa. The half-maximal inhibitory concentration (IC₅₀) of the heparin dodecasaccharide for anti-factor Xa activity is significantly lower than that of the commercially available fondaparinux sodium. Therefore, the anti-factor Xa activity of the novel heparin dodecasaccharide of the present disclosure is significantly superior to that of the heparin dodecasaccharide previously reported by Professor Jian Liu.
2. The anti-factor Xa activity of the heparin dodecasaccharide of the present disclosure can be effectively neutralized by protamine. For example, the neutralization rate of protamine against the anti-factor Xa activity of target compounds I-2 and I-10 exceeds 80%, which is comparable to that of unfractionated heparin. In contrast, the activity of commercially available fondaparinux sodium is almost completely unneutralizable. Although the heparin dodecasaccharide of the present disclosure does not contain four consecutive trisulfated disaccharide units, its anticoagulant activity can still be efficiently neutralized by protamine.
3. Minor modifications to the structure of the heparin dodecasaccharide of the present disclosure (e.g., sugar residues G, R₁~R₄) have less impact on its anti-factor Xa activity but significantly affect its protamine neutralization efficiency and pharmacokinetic profiles.
4. The heparin dodecasaccharide of the present disclosure can be effectively neutralized by protamine, making it suitable for the preparation of cost-effective and safer potent anticoagulant and antithrombotic drugs.
5. The two AT-binding sequences in the heparin dodecasaccharide of the present disclosure can independently activate AT, resulting in superior anti-factor Xa activity compared to the previously reported dodecasaccharides, which contain only a single AT-binding sequence. Its anti-factor Xa activity can be effectively neutralized by protamine, with a neutralization rate exceeding 80%. Additionally, the absence of multiple consecutive trisulfated disaccharide units avoids the pharmacokinetic defects (e.g., short half-life) and adverse reactions (e.g., heparin-induced thrombocytopenia, HIT) associated with such structural motifs.
6. The synthesis of the heparin dodecasaccharide of the present disclosure requires a minimum of 18 steps and a maximum of 21 steps, which is fewer than the 22-23 steps reported for existing dodecasaccharides. This reduction in synthesis steps lowers production costs, making it suitable for the preparation of cost-effective and safer potent anticoagulant and antithrombotic drugs.

### BRIEF DESCRIPTION OF THE DRAWINGS

FIG. 1A is the high-performance liquid chromatography (HPLC) chromatogram of the novel heparin dodecasaccharide I-2 prepared in Example 3;

FIG. 1B is the mass spectrum (B) of the novel heparin dodecasaccharide I-2 prepared in Example 3;

FIG. 1C is the high-performance liquid chromatography (HPLC) chromatogram of I-10 prepared in Example 3;

FIG. 1D is the mass spectrum of I-10 prepared in Example 3;

FIG. 2A is the ¹H NMR spectra of the novel heparin dodecasaccharide I-2 prepared in Example 3;

FIG. 2B is the HSQC spectra of the novel heparin dodecasaccharide I-2 prepared in Example 3;

FIG. 3 is the in vitro anti-factor Xa activity of the novel heparin dodecasaccharides I-2 and I-10 prepared in Example 3; and

FIG. 4 is the in vitro eutralization effect of protamine on the anticoagulant effect of the novel heparin dodecasaccharides I-2 and I-10 prepared in Example 3.

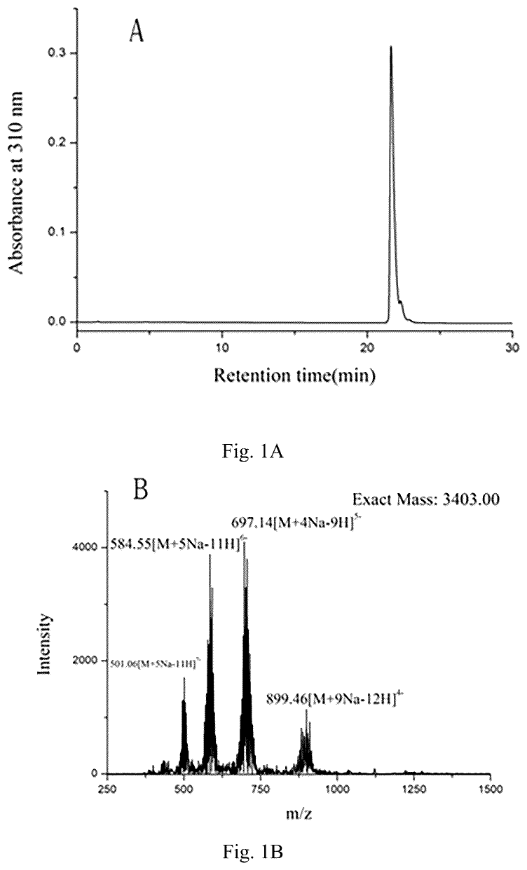

### DETAILED DESCRIPTION OF THE EMBODIMENTS

The present disclosure is further described and understood below in conjunction with specific embodiments. However, these examples should not be intended to limit the scope of protection of the present disclosure. The numbering of the target compounds in the following examples corresponds to that in Table 1. Unless otherwise specified, the drugs and reagents involved in the embodiments are commercially available products.

Commercialized kits referred to in the embodiments are commercially available products in the prior art.

### Example 1: Chemoenzymatic Synthesis of Heparin Pentasaccharide Intermediate Containing a Single IdoA2S Residue

500 mg of p-nitrophenyl-β-D-glucuronide (GlcA-PNP, 1) was dissolved in 200 mL of 50 mmol/L Tris-HCl buffer (containing 6 mmol/L MnCl₂, pH=7.2). To this solution, UDP-GlcNTFA (1.2 equiv.) and 5 mL of KfiA enzyme were added, and the mixture was stirred at room temperature overnight. The reaction was monitored by PAMN-HPLC under the following conditions: a gradient elution of 0→100% KH₂PO₄ over 45 min at a flow rate of 0.5 mL/min, with detection at 310 nm. When the yield reached ≥95%, the reaction was quenched by adjusting the pH to 2-3 with trifluoroacetic acid (TFA). The reaction mixture was purified using a C18 column (3.0×50 cm), with a gradient elution of methanol in water containing 0.1% TFA. The target fraction was collected, yielding the disaccharide backbone intermediate. The obtained disaccharide backbone intermediate was dissolved in 200 mL of 50 mmol/L Tris-HCl buffer (containing 6 mmol/L MnCl₂, pH=7.2). To this solution, UDP-GlcA (1.2 equiv.) and 5 mL of PmHS2 enzyme were added, and the mixture was stirred at room temperature overnight. The reaction was monitored by PAMN-HPLC until the yield reached ≥97%. The product was purified using a C18 column to obtain the trisaccharide backbone intermediate 3mer-1. Using 3mer-1 as the substrate, the above KfiA and PmHS2 reactions were repeated to obtain the pentasaccharide backbone intermediate 5mer-1. PAMN-HPLC analysis showed a purity of >82.5%, and ESI-MS confirmed its molecular weight (1181.09 Da), consistent with the calculated mass.

400 mg of the pentasaccharide backbone intermediate 5mer-1 was dissolved in 100 mL of deionized water and placed on ice. A 0.5 mol/L LiOH solution was added dropwise until the pH=12, and the mixture was kept on ice for 2 h. The reaction progress was monitored by PAMN-HPLC. After completion, the pH was adjusted to neutral with acetic acid, and 1 mol/L MES solution (pH=7.5) was added to achieve a final concentration of 50 mmol/L. To this solution, PAPS (2.5 equiv) and 3 mL of NST enzyme were added, and the mixture was stirred at room temperature overnight. The reaction was monitored by PAMN-HPLC. When the yield exceeded 95%, the reaction was quenched by adjusting the pH=4-5 with acetic acid. The product was purified using a Q Sepharose column (30×1.6 cm) at a flow rate of 3 mL/min, with a gradient elution of 0-100% 1 mol/L NaCl in 50 mmol/L NaAc buffer (pH=5). UV detection was performed at 260 nm and 310 nm. The target fraction was collected, desalted, and dried to obtain the N-sulfated heparin pentasaccharide 5mer-2. PAMN-HPLC analysis showed it has a purity of >78%, and ESI-MS confirmed its molecular weight (1149.17 Da), consistent with the calculated mass.

The heparin pentasaccharide 5mer-2 was dissolved in 100 mL of 50 mmol/L MES buffer (pH 7.0-7.5) containing 2 mmol/L CaCl₂ and C₅-epi enzyme. The reaction mixture was incubated in a 37 °C water bath for 2 h. Then, PAPS (~1.5 equiv), additional C₅-epi, and sufficient 2-OST enzyme were added, and the reaction was allowed to proceed at room temperature overnight. The reaction was monitored by PAMN-HPLC, and additional enzyme or PAPS was added if needed. After completion, the reaction mixture was purified using a Q-Sepharose strong anion exchange column (30×1.6 cm) to obtain the product 5mer-3. PAMN-HPLC analysis showed it has a purity of >95%, and ESI-MS confirmed its molecular weight (1129.27 Da), indicating the addition of one sulfate group compared to 5mer-2. Compound 5mer-3 is a heparin pentasaccharide containing a single IdoA2S residue.

The synthetic route for 5mer-3 is shown in formula II as follows:

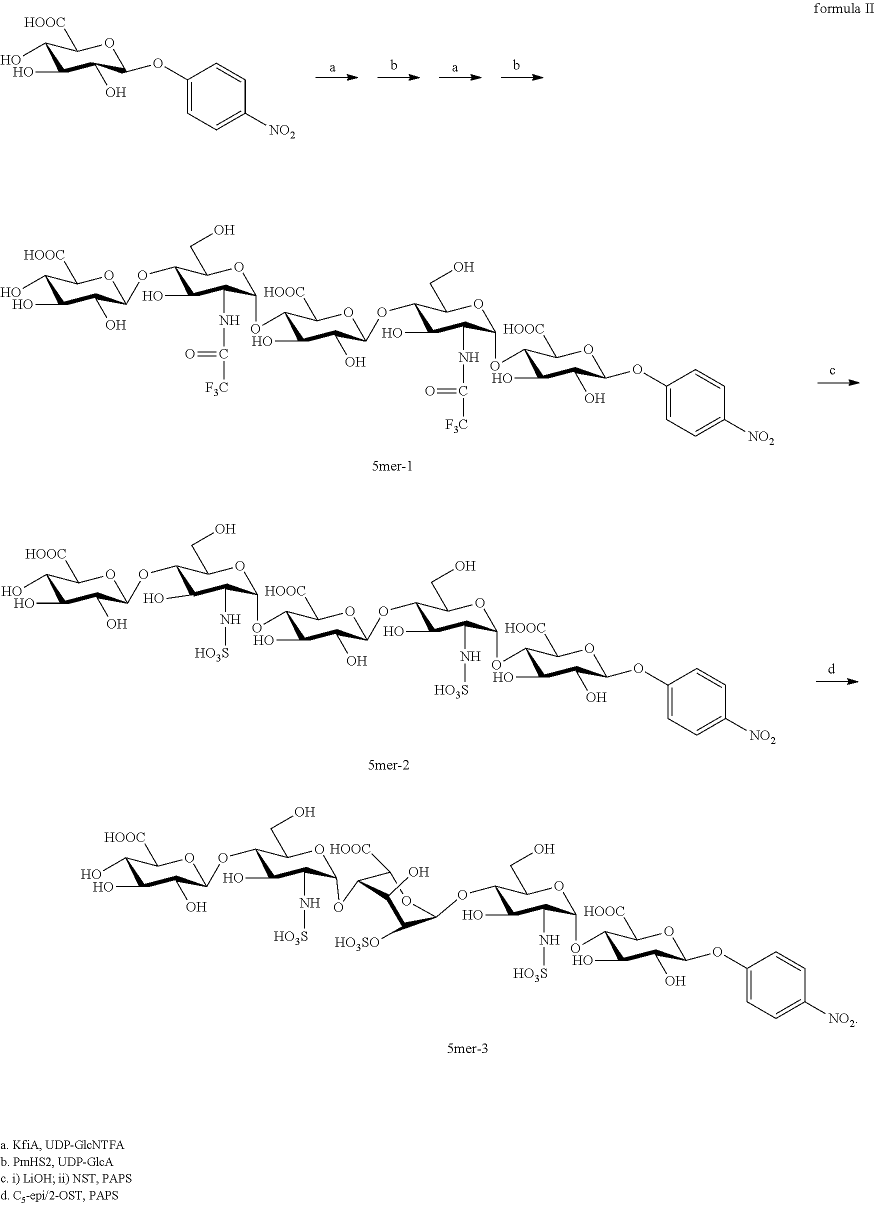

### Example 2: Chemoenzymatic Synthesis of Heparin Undecasaccharide Intermediates Containing Two and Three IdoA2S Residues

Referring to Example 1, 220 mg of the heparin pentasaccharide 5mer-3 containing a single IdoA2S residue was used as the starting material. Following the procedure described in Example 1, the glycosyl donor was replaced with UDP-GlcNAc, and the sugar chain was extended under the catalysis of KfiA. Subsequently, UDP-GlcA was used as the glycosyl donor, and the sugar chain was further extended under the catalysis of PmHS2. The enzymatic elongation of the sugar chain was then alternately performed using KfiA (with UDP-GlcNTFA as the glycosyl donor) and PmHS2 (with UDP-GlcA as the glycosyl donor) until heparin undecasaccharide 11mer-1 was formed. The product was purified using a Q Sepharose column (1×20 cm). The obtained 11mer-1 was treated with LiOH to remove the trifluoroacetyl groups, followed by N-sulfation catalyzed by NST, yielding heparin undecasaccharide 11mer-2. PAMN-HPLC analysis showed it has a purity of >99%, and ESI-MS confirmed its molecular weight (2442.16 Da), consistent with the calculated mass.

The heparin undecasaccharide 11mer-2 was subjected to co-catalysis by C₅-epi and 2OST, where a specific GlcA residue flanked by two GlcNS in the N-sulfated undecasaccharide chain was converted to 2-O-sulfated iduronic acid (IdoA2S). After a certain reaction time, the reaction mixture was purified using a Q Sepharose column (1×20 cm) to obtain 11mer-3, a heparin undecasaccharide containing two IdoA2S residues. If additional enzyme and PAPS were added to the reaction mixture and the reaction was continued until completion, followed by purification using a Q Sepharose column (1×20 cm) to yield 11mer-4, a heparin undecasaccharide containing three IdoA2S residues.

ESI-MS analysis confirmed that 11mer-3 had a molecular weight of 2522.21 Da, indicating the addition of one sulfate group compared to 11mer-2, corresponding to the incorporation of one additional IdoA2S residue;

ESI-MS analysis confirmed 11mer-4 had a molecular weight of 2602.40 Da, indicating the addition of two sulfate groups compared to 11mer-2, corresponding to the incorporation of two additional IdoA2S residues, consistent with the calculated mass.

The synthetic routes for 11mer-3 and 11mer-4 are shown in formula III as follows:

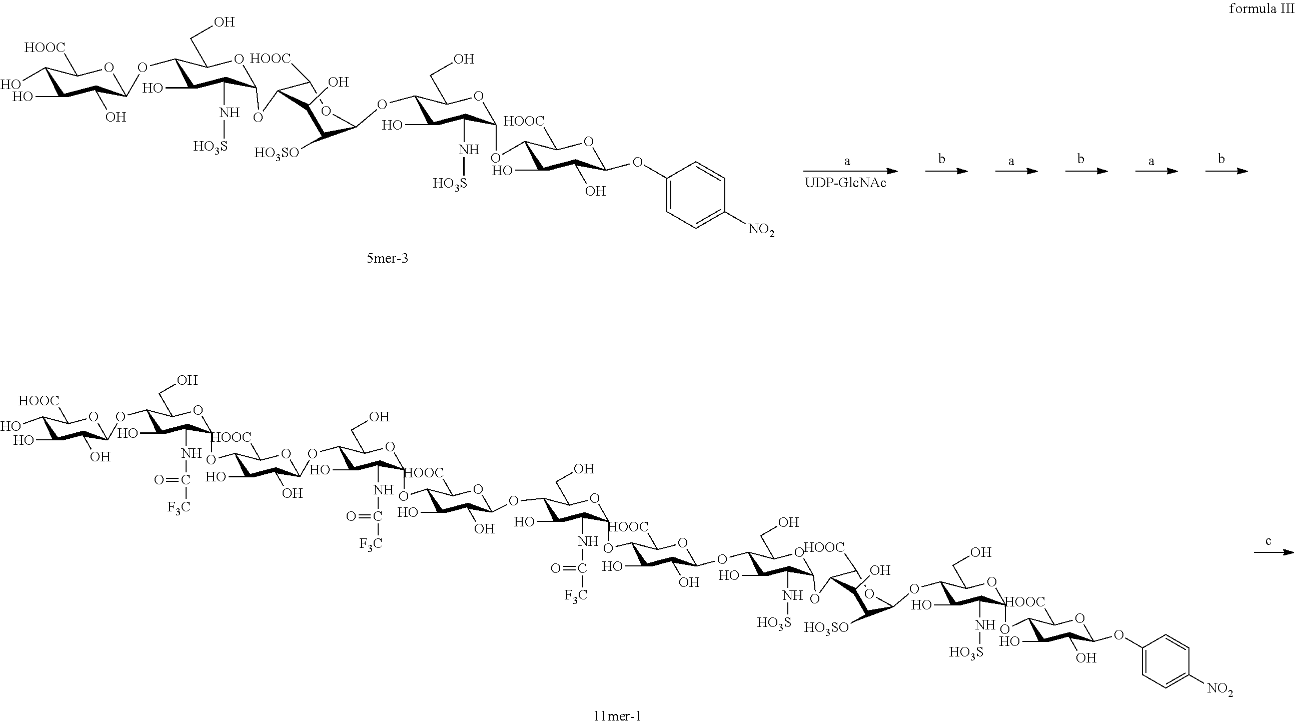

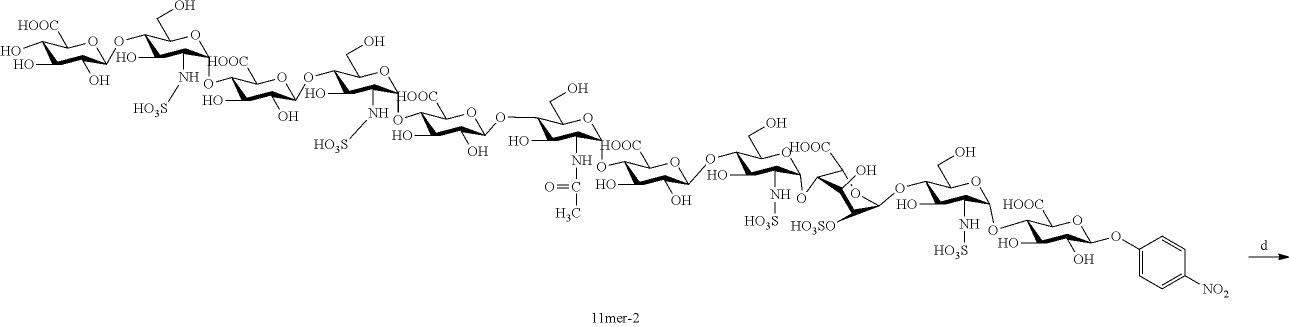

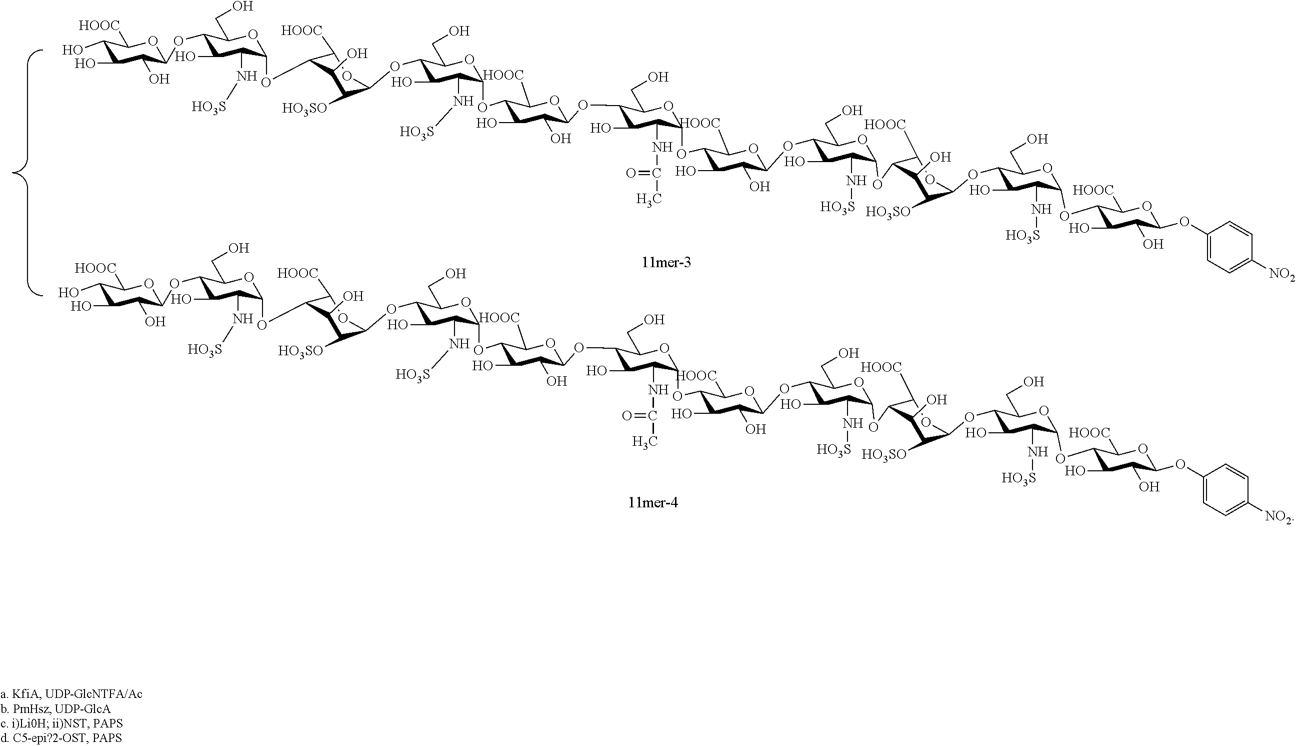

### Example 3: Preparation and Characterization of Heparin Dodecasaccharides I-2 and I-10 Containing Two AT-Binding Sequences

Using the undecasaccharides 11mer-3 and 11mer-4 from Example 2 as substrates, the sugar chain was extended enzymatically using KfiA following the method described in Example 1. The products were purified using a Q-Sepharose strong anion exchange column (1 cm×20 cm) to obtain the dodecasaccharide intermediates 12mer-1 and 12mer-2. Subsequently, the chemical removal of the trifluoroacetyl groups from GlcNTFA and enzymatic N-sulfation were performed as described above, yielding the dodecasaccharide intermediates 12mer-3 and 12mer-4. ESI-MS analysis confirmed the molecular weights of 2763.56 Da and 2843.39 Da, respectively, consistent with the theoretical molecular weight.

The dodecasaccharide substrates 12mer-3 and 12mer-4 were dissolved in 50 mmol/L MES buffer (pH=7.0-7.5). To this solution, PAPS (7 equiv.), 4 mL of 6-OST-1, and 4 mL of 6-OST-3 enzymes were added, and the reaction volume was adjusted to 140 mL. The reaction was carried out at 37 °C overnight. The reaction progress was monitored by SAX-HPLC, and additional enzyme or PAPS was added if needed. The chromatographic conditions were as follows: flow rate of 1 mL/min, gradient elution of 0-100% eluent B (50 mmol/L NaAc+2 mol/L NaCl, pH=5), with UV detection at 260 nm and 310 nm. When the conversion rate of 12mer-5 and 12mer-6 exceeded 99%, the reaction was quenched by adjusting the pH to 4-5 with acetic acid. The enzymes were removed by freeze-thaw cycles at −20 °C, and no further purification was required.

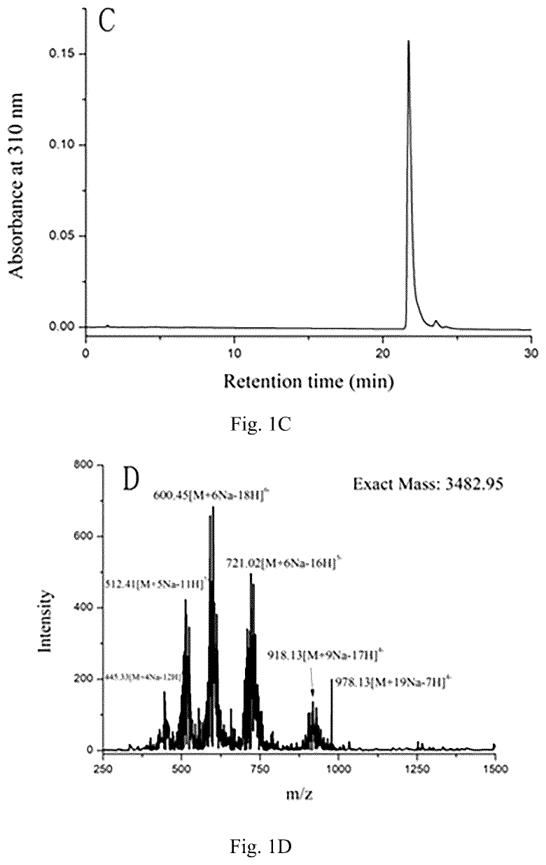

The reaction mixture was adjusted to pH=7.0-7.5, and PAPS (~2.5 equiv.) and 5 mL of 3-OST-1 enzyme were added, respectively. The reaction volume was adjusted to 200 mL, and the reaction was carried out at 37 °C overnight. The reaction was monitored by PAMN-HPLC until the substrate modification rate exceeded 99%. The reaction was quenched by adjusting the pH to 4-5 with dilute acetic acid, and the product was purified using a Q-Sepharose strong anion exchange column (1 cm×10 cm) to obtain I-2 with a purity of >92%. ESI-MS analysis confirmed its molecular weight of 3403.12 Da, consistent with the theoretical value. Similarly, I-10 was obtained with a purity of >97%, and ESI-MS analysis confirmed a molecular weight of 3482.97 Da, consistent with the calculated mass. The NMR spectrum (600 MHz, D₂O) of I-2 is shown in FIG. 2, and the structure is as expected.

The synthetic route is shown in formula IV as follows:

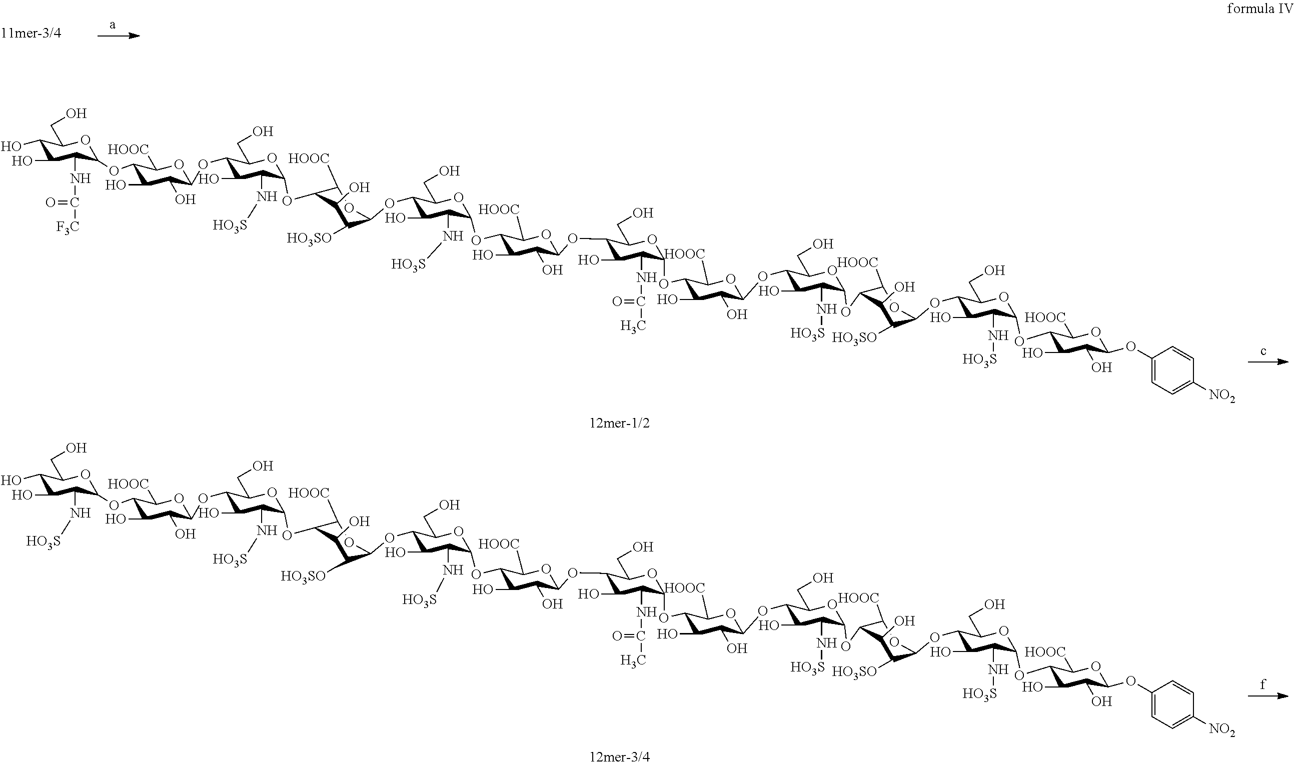

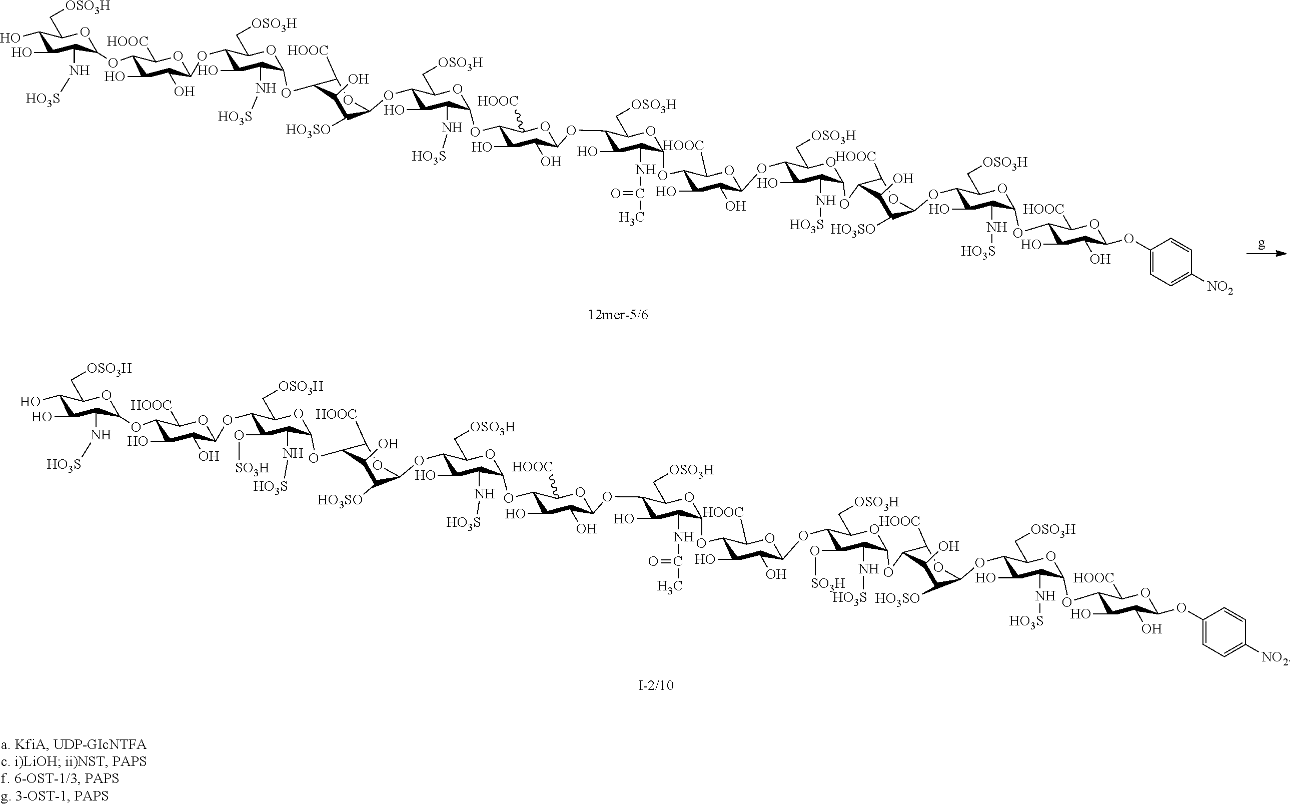

### Example 4: In Vitro Anticoagulant Activity Assay of Heparin Dodecasaccharides I-2 and I-10

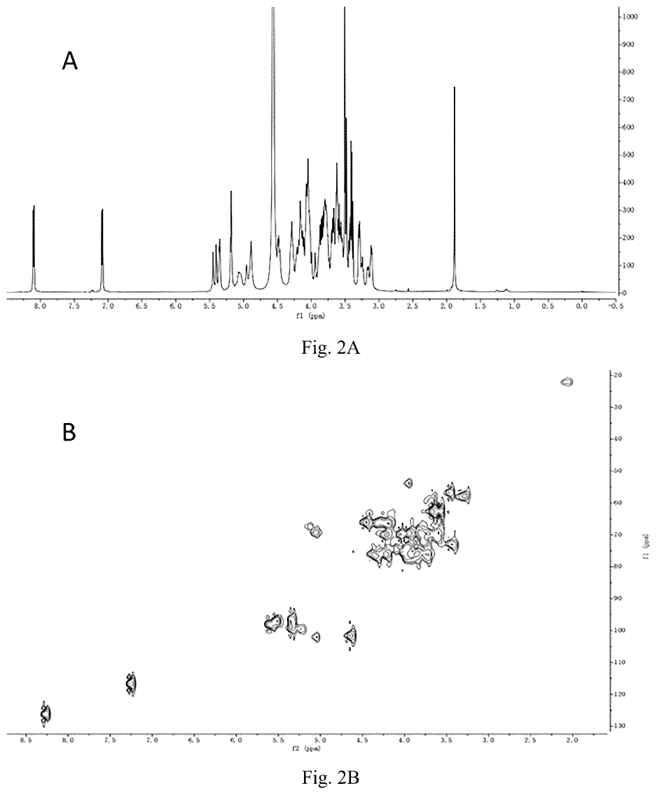

The anti-factor Xa (FXa) activity of the novel heparin dodecasaccharides I-2 and I-10 prepared in the present disclosure was determined using a commercially available chromogenic substrate assay kit. The IC₅₀ values for the anti-FXa activity of I-2 and I-10 were measured to be 16.77 ng/mL (4.34 nmol/L) and 18.03 ng/mL (4.54 nmol/L), respectively. Under the same conditions, the IC₅₀ values for unfractionated heparin (UFH) and fondaparinux sodium (Arixtra) were 139 ng/mL and 12.63 ng/mL (7.3 nmol/L), respectively. When expressed in molar concentration, the IC₅₀ values of heparin dodecasaccharides I-2 and I-10 for anti-FXa activity were significantly lower than that of fondaparinux sodium. The test results, as shown in FIG. 3, demonstrated that the novel heparin dodecasaccharides I-2 and I-10 prepared in the present invention exhibited no significant anti-factor IIa activity (data not shown). Therefore, the novel heparin dodecasaccharides I-2 and I-10 prepared in the present disclosure are specific inhibitors of factor Xa.

### Experimental Example 5: Neutralization Effect of Protamine on the Anticoagulant Activity of Heparin Dodecasaccharides I-2 and I-10

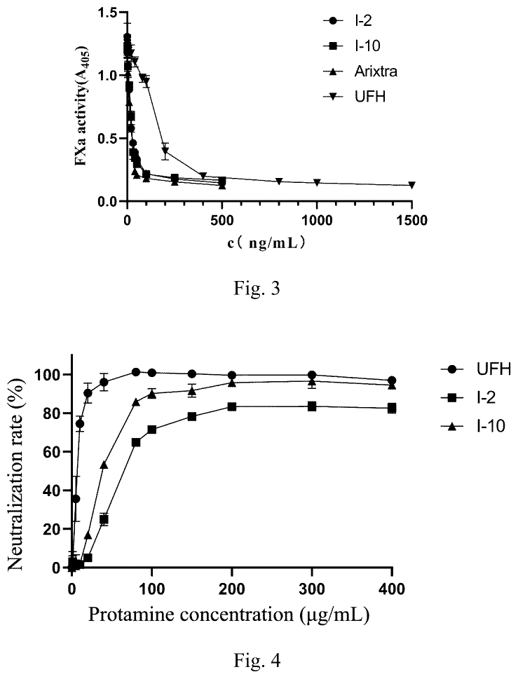

Using the chromogenic substrate assay, the effect of different concentrations of protamine on the anti-Factor Xa (FXa) activity of the novel heparin dodecasaccharides I-2 and I-10 was evaluated. The results demonstrated that, similar to unfractionated heparin (UFH), the anti-FXa activity of the novel heparin dodecasaccharide I-10 in vitro could be completely reversed by protamine. The in vitro anti-FXa activity of the novel heparin dodecasaccharide I-2 could be reversed by >80% with protamine, as shown in FIG. 4. Therefore, the heparin dodecasaccharides I-2 and I-10 prepared in the present disclosure are novel heparin molecules whose anticoagulant activity can be neutralized by protamine.

As shown in Example 4 and FIG. 3, the IC₅₀ values for the anti-FXa activity of I-2 and I-10 were 4.34 nmol/L and 4.54 nmol/L, respectively, indicating minimal difference. However, as shown in Example 5 and FIG. 4, the neutralization rate of protamine against the anticoagulant activity of I-2 exceeded 80%, while that of I-10 exceeded 90%, showing a significant difference. Thus, the replacement of the sugar residue G from GlcA to IdoA2S had a minor impact on the anti-FXa activity of the heparin dodecasaccharides but significantly affected their neutralization efficiency by protamine.

What is claimed is:

1. A heparin dodecasaccharide molecule comprising two AT-binding sequences and lacking consecutive multiple trisulfated disaccharide (IdoA2S-GlcNS6S) units, or pharmaceutically acceptable salts thereof, having the structure shown in formula I. 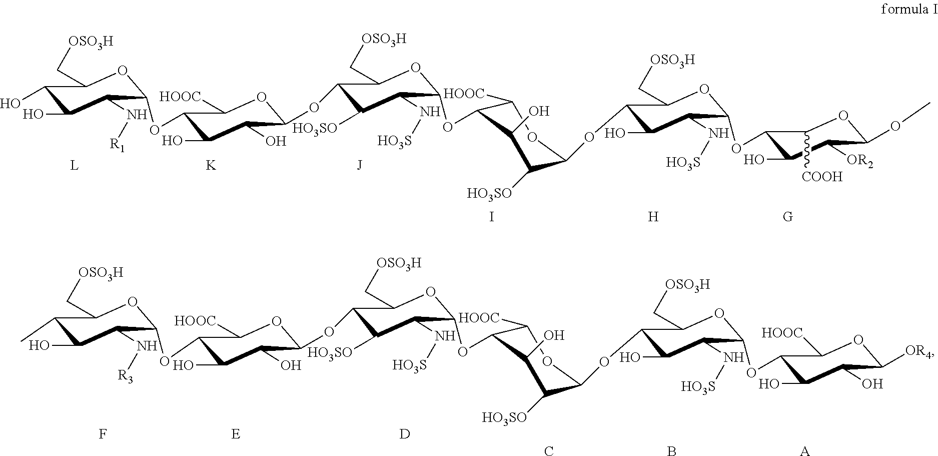
wherein R₁ and R₃ are sulfo groups (—SO₃H) or acetyl groups (—COCH₃); R₂ is sulfo groups (—SO₃H) or hydrogen (—H);
R₄ is selected from phenyl or substituted phenyl group with characteristic UV absorption, or aromatic heterocycle or substituted aromatic heterocycle;
sugar residue G is glucuronic acid (GlcA) or iduronic acid (IdoA) residue.

2. The heparin dodecasaccharide molecule or pharmaceutically acceptable salts thereof according to claim 1, wherein the substituent on the substituted phenyl or substituted aromatic heterocycle is nitro, halogen, hydroxyl, or trifluoromethyl.

3. The heparin dodecasaccharide molecule according to claim 1, wherein the heparin dodecasaccharide molecule is selected from the group consisting of compound I-1 having the structure of the formula I whose G=GlcA, R₁=R₃=—SO₃H, R₂=—H, R₄=-PNP; compound I-2 having the structure of the formula I whose G=GlcA, R₁=—SO₃H, R₃=-Ac, R₂=—H, R₄=-PNP; compound I-3 having the structure of the formula I whose G=GlcA, R₁=-Ac, R₃=—SO₃H, R₂=—H, R₄=-PNP; compound I-4 having the structure of the formula I whose G=GlcA, R₁=R₃=-Ac, R₂=—H, R₄=-PNP; compound I-5 having the structure of the formula I whose G=GlcA, R₁=R₂=R₃=—SO₃H, R₄=-PNP; compound I-6 having the structure of the formula I whose G=GlcA, R₁=R₂=—SO₃H, R₃=-Ac, R₄=-PNP; compound I-7 having the structure of the formula I whose G=GlcA, R₁=-Ac, R₂=R₃=—SO₃H, R₄=-PNP; compound I-8 having the structure of the formula I whose G=GlcA, R₁=R₃=-Ac, R₂=—SO₃H, R₄=-PNP; compound I-9 having the structure of the formula I whose G=IdoA, R₁=R₂=R₃=—SO₃H, R₄=-PNP; compound I-10 having the structure of the formula I whose G=IdoA, R₁=R₂=SO₃H, R₃=-Ac, R₄=-PNP; compound I-11 having the structure of the formula I whose G=IdoA, R₁=-Ac, R₂=R₃=—SO₃H, R₄=-PNP; and compound I-12 having the structure of the formula I whose G=IdoA, R₁=R₃=-Ac, R₂=—SO₃H, R₄=-PNP.

4. A method for preparing the heparin dodecasaccharides containing two AT-binding sequences and lacking consecutive multiple trisulfated disaccharide units according to claim 1, wherein the method uses a glucuronic acid (GlcA) derivative with a reducing end covalently linked to an R₄ group as a starting substrate, and comprises the following glycosyltransferase-catalyzed reactions (steps a, b) repeated at least once; and a combination with four or five steps from chemoenzymatic modification reactions (steps c, d, e, f, g);
at step a: under the catalysis of N-acetylglucosaminyltransferase (KfiA) or Heparosan Synthase 2 (PmHS2), using UDP-GlcNTFA or UDP-GlcNAc as the glycosyl donor, the GlcNTFA or GlcNAc residue of the glycosyl donor is transferred via an α-1,4-glycosidic bond to the non-reducing end GlcA of the substrate, yielding an intermediate compound;
at step b: under the catalysis of PmHS2, using UDP-GlcA as the glycosyl donor, the GlcA residue of the glycosyl donor is linked via a β-1,4-glycosidic bond to the non-reducing end glucosamine (GlcNTFA or GlcNAc) of the substrate, yielding an intermediate compound;
at step c: The heparin intermediate is incubated in a mild alkaline aqueous solution on ice, where the GlcNTFA residues of the sugar chain undergo complete N-detrifluoroacetylation to form GlcNH₂, followed by converting to GlcNS under the catalysis of N-sulfotransferase (NST), yielding an N-sulfated intermediate;
at step d: under the co-catalysis of C₅-epimerase (C₅-epi) and 2-O-sulfotransferase (2OST), specific GlcA residues between two GlcNS residues or between GlcNS (non-reducing end) and GlcNAc in the N-sulfated product are converted to 2-O-sulfated iduronic acid (IdoA2S), yielding an intermediate containing IdoA2S residues;
at step e: under the catalysis of 2OST alone, specific GlcA residues between two GlcNS residues or between GlcNS (non-reducing end) and GlcNAc in the substrate are converted to 2-O-sulfated glucuronic acid (GlcA2S), yielding an intermediate containing GlcA2S residues;
at step f: under the co-catalysis of 6-O-sulfotransferase 1 and 3 (6OST1, 6OST3), all GlcNS or GlcNAc residues in the sugar chain are sulfated at the 6-OH position to form GlcNS6S or GlcNAc6S, yielding a 6-O-sulfated intermediate; and
at step g: under the catalysis of 3-O-sulfotransferase 1 (3OST1), the 3-OH position of GlcNS6S between GlcA and IdoA2S in the sugar chain is sulfated (GlcNS6S3S), yielding the final target compound.

5. The method according to claim 4, wherein the starting substrate is p-nitrophenyl-β-D-glucuronide (GlcA-PNP); in step a, the N-acetylglucosaminyltransferase (KfiA) and Heparosan synthase 2 (PmHS2) are recombinantly expressed in Escherichia coli. KfiA is derived from Escherichia coli K5, and PmHS2 is derived from Pasteurella multocida;
at steps a and b, the enzymatic reactions are carried out at 20 °C-37 °C in a buffer of 50 mmol/L Tris-HCl containing 6 mmol/L MnCl₂, pH 7.0-7.5; the amounts of enzyme and substrate as well as the reaction time, are not limited; the enzymatic reaction mixture is purified using reverse-phase C18 or anion-exchange column chromatography to obtain the intermediate compound; The glycosyl donor is added in an amount of at least 1.2 equivalents relative to the substrate.

6. The method according to claim 4, wherein at steps c, d, e, f, and g, the heparin-modifying enzymes NST, C₅-epi, 2OST, 6OST1, 6-OST3, and 3OST1 are recombinantly expressed using E coli, yeast, or insect cells; the heparin-modifying enzymes NST, 2OST, 6OST1, 6-OST3, and 3OST1 all use 3′-phosphoadenosine-5′-phosphosulfate (PAPS) as the sulfate donor; the buffer used for each enzymatic reaction is 50 mmol/L 2-(N-morpholino)ethanesulfonic acid (MES), pH 7.0-7.5, and the reaction temperature is maintained at 20 °C-37 °C; the amounts of enzyme and heparin intermediate substrate, as well as the reaction time, are not limited; the reaction mixture is purified using anion-exchange column chromatography to obtain the product; in steps c, d, e, f, and g, the sulfate donor is added in an amount of 1.5-10 equivalents relative to the substrate.

7. The method according to claim 4, wherein the method is selected from one of the following synthetic routes:
a→b→a→b→c→d→a→b→c→f→a→b→a→b→c→d-a-c-f-g for compound I-1,
a→b→a→b→c→d→a→b→f→a→b→a→b→c→d→a→c→f→g or a→b→a→b→c→d→a→b→a→b→a→b→c→d→a→c→f→g for compound I-2,
a→b→a→b→c→d→a→b→c→f→a→b→a→b→c→d→a→f→g for compound I-3,
a→b→a→b→c→d→a→b→f→a→b→a→b→c→d→a→f→g for compound I-4,
a→b→a→b→c→d→f→a→b→a→b→c→e→a→b→c→d→a→c→f→g for compound I-5,
a→b→a→b→c→d→f→a→b→a→b→c→e→a→b→c→d→a→c→f→g for compound I-6
a→b→a→b→c→d→f→a→b→a→b→c→e→a→b→c→d→a→f→g for compound I-7,
a→b→a→b→c→d→f→a→b→a→b→c→e→a→b→c→d→a→f→g for compound I-8,
a→b→a→b→c→d→f→a→b→a→b→a→b→c→d→a→c→f→g for compound I-9,
a→b→a→b→c→d→f→a→b→a→b→a→b→c→d→a→c→f→g or a→b→a→b→c→d→a→b→a→b→a→b→c→d→a→c→f→g for compound I-10,
a→b→a→b→c→d→f→a→b→a→b→a→b→c→d→a→f→g for compound I-11 and
a→b→a→b→c→d→f→a→b→a→b→a→b→c→d→a→f→g for compound I-12.

8. The method according to claim 4, wherein the synthetic route is as follows:
a→b→a→b→c→d→a→b→a→b→a→b→c→d→a→c→f→g.

9. A process for preparing a heparin dodecasaccharide containing two AT-binding sequences and lacking consecutive multiple trisulfated disaccharides of claim 1, comprising the following steps:
i) under the catalysis of N-acetylglucosaminyltransferase (KfiA) or Heparosan synthase 2 (PmHS2), using UDP-GlcNTFA as the glycosyl donor, the GlcNTFA residue of the glycosyl donor is transferred via an α-1,4-glycosidic bond to the non-reducing end GlcA of the substrate, yielding a disaccharide backbone intermediate;
ii) under the catalysis of PmHS2, using UDP-GlcA as the glycosyl donor, the GlcA residue of the glycosyl donor is linked via a β-1,4-glycosidic bond to the non-reducing end GlcNTFA of the disaccharide backbone, yielding a trisaccharide backbone intermediate;
iii) steps i) and ii) are repeated to extend the sugar chain, yielding a pentasaccharide backbone intermediate;
iv) the pentasaccharide backbone intermediate is incubated in a mild alkaline aqueous solution on ice, where the GlcNTFA residues of the sugar chain undergo complete N-detrifluoroacetylation to form GlcNH₂; subsequently, these GlcNH₂ residues are converted to GlcNS under the catalysis of N-sulfotransferase (NST), yielding an N-sulfated pentasaccharide intermediate;
v) under the co-catalysis of C5-epimerase (C5-epi) and 2-O-sulfotransferase (2OST), a specific GlcA residue between two GlcNS residues in the N-sulfated pentasaccharide intermediate is converted to 2-O-sulfated iduronic acid (IdoA2S), yielding a heparin pentasaccharide containing one IdoA2S residue;
vi) referring to step i), the glycosyl donor is replaced with UDP-GlcNAc, and the sugar chain is extended under the catalysis of KfiA or PmHS2 to yield a hexasaccharide intermediate; using the hexasaccharide intermediate as the substrate, the sugar chain is extended to a heptasaccharide by referring to step ii), yielding a heptasaccharide intermediate; using the heptasaccharide intermediate as the substrate, steps 1) and 2) are alternately repeated to further extend the sugar chain, yielding an undecasaccharide intermediate;
vii) the undecasaccharide intermediate is incubated in a mild alkaline aqueous solution on ice, where the GlcNTFA residues of the sugar chain undergo complete N-detrifluoroacetylation to form GlcNH2; subsequently, these GlcNH2 residues are converted to GlcNS under the catalysis of NST, yielding an N-sulfated undecasaccharide;
viii) under the co-catalysis of C₅-epi and 2OST, a specific GlcA residue between two GlcNS residues in the N-sulfated undecasaccharide is converted to IdoA2S, yielding a heparin undecasaccharide containing two IdoA2S residues; By adding an appropriate amount of enzyme and substrate and extending the reaction time, a specific GlcA residue between GlcNS (non-reducing end) and GlcNAc in the undecasaccharide is further converted to IdoA2S, yielding a heparin undecasaccharide containing three IdoA2S residues;
ix) in step viii), the heparin undecasaccharide containing two or three IdoA2S residues is extended by repeating step 1), yielding a heparin dodecasaccharide containing two or three IdoA2S residues;
x) the heparin dodecasaccharide containing two or three IdoA2S residues is incubated in a mild alkaline aqueous solution on ice, where the newly introduced GlcNTFA residue undergoes N-detrifluoroacetylation to form GlcNH2; subsequently, this GlcNH2 residue is converted to GlcNS under the catalysis of NST, yielding an N-sulfated heparin dodecasaccharide containing two or three IdoA2S residues;
xi) under the co-catalysis of 6-O-sulfotransferase 1 and 3 (6OST1, 6OST3), all GlcNS or GlcNAc residues in the sugar chain of the N-sulfated heparin dodecasaccharide containing two or three IdoA2S residues are sulfated at the 6-OH position to form GlcNS6S or GlcNAc6S, yielding two types of 6-O-sulfated heparin dodecasaccharides;
xii) under the catalysis of 3-O-sulfotransferase 1 (3OST1), the 3-OH position of GlcNS6S between GlcA and IdoA2S in the sugar chain of the two types of 6-O-sulfated heparin dodecasaccharides is sulfated (GlcNS6S3S), yielding the target compound I-2 containing two IdoA2S residues or the target compound I-10 containing three IdoA2S residues.

10. The process according to claim 9, wherein the heparin dodecasaccharides containing two AT-binding sequences and lacking consecutive multiple trisulfated disaccharide units is used for the preparation of anticoagulant and antithrombotic drug, wherein the anticoagulant and antithrombotic drug, comprising the heparin dodecasaccharide containing two AT-binding sequences and lacking consecutive multiple trisulfated disaccharide units, and one or more pharmaceutically acceptable carriers or excipients.
